(19) United States
(12) Patent Application Publication
(10) Pub. No.: US 2025/0340705 A1
(43) Pub. Date: Nov. 6, 2025

(54) RUBBER ARTICLES, RUBBER GLOVES CONTAINING NANOPARTICLES WITH ACTIVE SUBSTANCES, AND ASSOCIATED METHODS THEREOF

(71) Applicant: GODEVA PTE. LTD., Singapore (SG)

(72) Inventor: Boon Joo TENG, MELAKA (MY)

(73) Assignee: GODEVA PTE. LTD., SINGAPORE (SG)

(21) Appl. No.: 18/854,414

(22) PCT Filed: Jul. 13, 2022
(86) PCT No.: PCT/MY2022/050062

§ 371 Date: Oct. 4, 2024

(30) Foreign Application Priority Data
Apr. 4, 2022 (MY) PI2022001772

(51) Int. Cl. C08J 5/02; A41D 19/00

(52) U.S. Cl. CPC C08J 5/02; A41D 19/0062; C08J 2307/02; C08J 2321/02

(57) ABSTRACT

A method for preparing a rubber article by using nano size particles includes the steps of, providing latex from a latex source; mixing the latex with rubber chemicals to produce a compounded latex; and, using the compounded latex to prepare the rubber article via an article dipping or a molding process. In use, the rubber article is a rubber glove. Those of ordinary skills in the art will appreciate that the present disclosure and various embodiments of the present invention are disclosed herein in a manner such that the novel and unique aspects may be implemented for rubber gloves, different types of one or more rubber articles, and the like.

### TECHNICAL FIELD

Embodiments of the present invention generally relate to the field of rubber articles, and, more particularly, to rubber articles, rubber gloves containing nanoparticles with active substances, and associated methods thereof.

### BACKGROUND ART

Generally, healthcare industries, semiconductor industries, food industries and others use gloves or articles in different workplaces as a standard precaution to prevent users from harmful substances. For the record, healthcare industries have been utilising protective gloves or articles to keep them from cross-contamination of pathogens. At the same time, protective gloves or articles are used by electricians to preclude contamination and short circuit due to electrostatic and other on-site damages. Additionally, food handlers require wearing gloves or articles to evade the risk of bacterial contamination and hygiene. These gloves or articles work as a shielding barrier against several perilous damages and mitigate the risk of contamination. As a result, these protective gloves or articles ought to deliver all-around protection effectively.

Generally, latex gloves made of natural rubber work as an effectual shield for protection. Besides, antimicrobial technology is applied on gloves or articles to constrain harmful bacteria, mould, and mildew growth and reproduction. This antimicrobial property is a combination of one or more ingredients, including but not limited to Metal Oxide and antimicrobial drugs. The molecules present on the glove kill harmful microorganisms.

Additionally, prospective testing has been performed that reveals that nanoparticles infused gloves or articles can target bacteria effectively. The compact molecular structure yields better pore size, giving an excellent thermal insulation property where the heat from the external is redundant. The smaller pore size rubber matrix yields a good chemo drug resistance where it protects against chemo-drug permeability. But conventional articles or gloves lack active nanoparticle substances and the antimicrobe technology are usually coated on the surface of the glove which dissipate at short time.

Consequently, as a result, the gloves or articles' kill properties are counterproductive in antimicrobial action, electrostatic resistance, heat protection, and chemo drug resistance. In addition, the production of this protective glove or article has significantly increased due to increased requirements in industries. Most of the time, depending on the industry type, the user must wear this gloves or articles actively during the work process. The durability of gloves relates to a glove's or article's longevity in killing harmful microorganisms. In this context, typical articles or gloves generally have a specific use of duration, usually lasting for a short period of time. This affects the integrity and entire purpose of the gloves and articles.

Furthermore, the conventional gloves or articles has antimicrobial technology application which mostly coated on surfaces that are fast deteriorated or leached out upon exposure and/or worn by the user; hence the protection level on the user is less effective, the risk of contamination is raised and causes a biological hazard to both healthcare personnel and patient. Additionally, the conventional articles or gloves have electrostatic resistivity of certain ohm, which may or may not resist the electrostatic in an electrical component assembly line. Furthermore, the use in food industries is single-use products that can cross-contamination from food to consumer or vice versa.

However, the present articles or gloves utilized the tested nanotechnology into their invention. The nanoparticles active substances provide the compact molecular structure compared to articles or gloves without nanoparticles active substances. This present article or glove has an effective kill rate on microorganisms as well as provides an excellent thermal insulation property where the heat from the external is redundant. The nanoparticle along with other components infused in rubber latex gloves or articles form an effective component to defeat bacterial and cross contamination.

Accordingly, there remains a need in the art for innovative, novel, efficient solutions for providing complete and adequate protection against cross-contamination and injuries by using latex gloves along with nanoparticles active substance suffused. Specifically, there remains a need in the art for innovative, novel, cost-effective and efficient solutions providing rubber articles, rubber gloves containing nanoparticles with active substances, and associated methods thereof.

### SUMMARY OF THE INVENTION

It is an object of the invention to provide rubber articles, rubber gloves containing nanoparticles with active substances, and associated methods thereof.

In one embodiment, a method for preparing a rubber article by using nano size particles includes the steps of, providing latex from a latex source; mixing the latex with rubber chemicals to produce a compounded latex; and, using the compounded latex to prepare the rubber article via an article dipping or a molding process. In use, the rubber article is a rubber glove. Those of ordinary skills in the art will appreciate that the present disclosure and various embodiments of the present invention are disclosed herein in a manner such that the novel and unique aspects may be implemented for rubber gloves, different types of one or more rubber articles, and the like.

The embodiments of the present disclosure have several features, no single one of which is solely responsible for their desirable attributes. Without limiting the scope of the present embodiments as expressed by the claims that follow, their more prominent features will now be discussed briefly. After considering this discussion, and particularly after reading the section entitled “Detailed Description”, one will understand how the features of the present embodiments provide advantages, which include rubber articles, rubber gloves containing nanoparticles with active substances, and associated methods thereof.

### DETAILED DESCRIPTION OF PREFERRED EMBODIMENTS

Various embodiments of the present invention are disclosed herein below, which relate to rubber articles, rubber gloves containing nanoparticles with active substances, and associated methods thereof. Generally, gloves can protect your hands from a variety of hazards in the workplace or at home. Such jobs are frequently carried out in fluid settings, which necessitate not only protection against materials such as water, aqueous solutions of various degrees of alkalinity or acidity, oil, gasoline, or similar fluids, but also the ability to grip and securely hold or manipulate an object. For these goals, the gloves should be comfortable, flexible, breathable, and have a grip surface capable of stable grasp even when exposed to materials with lubricity that would impair one's ability to grip an object securely.

Particularly, hands come in a variety of sizes and shapes, depending on the person. Unfortunately, a standardised medical glove does not fit the hand correctly, and as a result, medical professionals are hesitant to use it since it may cause medical treatment, which must be carried out precisely, to fail.

Additionally, SARS Covid, Hepatitis B, AIDS (acquired immune deficiency syndrome), and other diseases, as well as dangerous chemicals and fertilizers, have recently piqued people's curiosity. While being exposed to such deadly diseases, medical personnel are required to provide medical care, such as surgical operations or the like and industry people need protective equipment. As a result, medical personnel and other industries require the use of a protective glove. The traditional gloves, unfortunately, does not suit the hand, particularly the fingers. As a result, industries' personnel are unable to provide treatment without experiencing worry.

Particularly, the novel infused nanoparticle articles or gloves as disclosed herein have improved characteristics and can stay dispersed in a liquid for longer periods of time. In use, when a coating layer is generated on the nanoparticles, the slurry have tiny particle size and excellent dispersion capabilities in a polymer matrix. These rubber articles or gloves with nano particles active substances are manufactured by polymer latexes either Natural Rubber latex or Synthetic Latex with nanoparticles active substances' chemical and at least one or combination of rubber chemicals such as Sulphur, Zinc Oxide, ZDEC, ZDBC. The polymer to use for producing the rubber articles or gloves are Natural Rubber Latex and Synthetic Latex such as Nitrile Butadiene Rubber (NBR), but not limited to Polyisoprene Rubber, Polychloroprene Rubber, Polyvinyl Chloride etc.

There are the various embodiment of the present invention that are disclosed herein below that relate to the Active material with nano size particles infuses into a rubber matrix for barrier protection. Moreover, Rubber articles or gloves containing nanoparticles active substances to be worn by healthcare personnel in healthcare industries but also in electrical industries, food industries, or any other application.

In accordance with an embodiment of the present invention, the rubber articles or gloves with nano particles active substances are manufactured by polymer latexes either Natural Rubber latex or Synthetic Latex with nanoparticles active substances' chemical and at least one or combination of rubber chemicals such as Sulphur, Zinc Oxide, ZDEC, ZDBC. In use, the process of manufacturing the products involves latex mixing, articles or gloves dipping or moulding process, post processing with or without surface retreatment to enhance the surface morphologies for better handling of instruments.

In accordance with an embodiment of the present invention, the articles or gloves consist of nanoparticle sizes of chemicals. In operation, the invention of the nanoparticles with active substances onto the articles or gloves yield the compact structure and which chemically strengthen the rubber film compared to present articles.

In accordance with an embodiment of the present invention, rubber articles or gloves with the nano size particles leading to effective antimicrobial action properties help to prevent cross-contamination of bodily fluid which risk exposure to pathogens. In use, it has an effective kill rate on microorganisms such as Escherichia coli, Staphylococcus Aureus, Pseudomonas Aeruginosa, but not limited to Covid virus.

In accordance with an embodiment of the present invention, the nanoparticles active substance has shown better durability in kill rate where the result of Inoculum count is maintained <10 cfu/ml after 0 min, 5 min. and 30 min. contact with the tested Organism.

In accordance with an embodiment of the present invention, rubber articles or gloves with the nano size particles leading to effective electrostatic resistance. In operation, electrostatic resistivity resists the electrostatic in electrical component assembly line.

In accordance with an embodiment of the present invention, rubber articles or gloves with the nano size particles leading to effective heat protection. In use, the compact molecular structure yields better pore size which gives a good thermal insulation properties where the heat from external is redundant.

In accordance with an embodiment of the present invention, rubber articles or gloves with the nano size particles leading to effective chemo drug resistance. In operation, chemo drug resistance Rubber articles or gloves to be worn by healthcare personnel, during preparing chemo-drug and/or handling patients with chemotherapy. In accordance with an embodiment of the present invention, rubber articles or gloves produced with nano particles active substances yield strong elasticity products. In use, they also have a tighter fit and more stretch, making them more comfortable to wear for longer periods of time and enabling enhanced dexterity.

In accordance with an embodiment of the present invention, rubber articles or gloves produced from rubber polymer such as natural rubber, synthetic rubber like Nitrile Butadiene Rubber (NBR). In use, Rubber articles or gloves produced from rubber polymer such as natural rubber, synthetic rubber, but not limited to Polyisoprene Rubber, Polychloroprene Rubber, Polyvinyl Chloride etc. helps in making various methods for production of articles or gloves.

In accordance with an embodiment of the present invention, a method for preparing a rubber article by using nano size particles includes the steps of, providing latex from a latex source; mixing the latex with rubber chemicals to produce a compounded latex; and, using the compounded latex to prepare the rubber article via an article dipping or a molding process. In use, the rubber article is a rubber glove. Those of ordinary skills in the art will appreciate that the present disclosure and various embodiments of the present invention are disclosed herein in a manner such that the novel and unique aspects may be implemented for rubber gloves, different types of one or more rubber articles, and the like.

In accordance with an embodiment of the present invention, the latex source is natural rubber latex. In use, the latex source may be synthetic latex as well.

In accordance with an embodiment of the present invention, the rubber chemicals include a plurality of nano size particles of an active substance. In use, the method further includes the step of post-processing the rubber article with or without surface retreatment to enhance surface morphologies for better handling of instruments. Also, the method further includes the step of using the plurality of nano size particles with an active substance onto the rubber article to yield a compact structure of a rubber matrix.

In accordance with an embodiment of the present invention, the method further includes the step of infusing nanoparticles active substances into a rubber matrix which contains one or at least one or combination of a plurality of ingredients, such as alkanolamines, amino acids, surfactants, plant-based derivatives in a base of colloids, micelles to give anti-microbial properties, and the like. In use, the rubber chemicals are selected from a group including at least one or combination of chemicals including Sulphur, Zinc Oxide, ZDEC, ZDBC, any other curative chemicals, crossliner, antioxidant, colorant, and the like.

In accordance with an embodiment of the present invention, the method further includes the step of producing the rubber article with nanoparticles active substances leading to effective electrostatic resistance. In use, the method further includes the step of producing the rubber article with nanoparticles active substances leading to effective heat protection. Also, the method further includes the step of producing the rubber article with nanoparticles active substances leading to effective chemo drug resistance. Moreover, the method further includes the step of producing the rubber article with nanoparticles active substances by using polymer including Natural Rubber Latex and/or Synthetic Latex, such as Nitrile Butadiene Rubber (NBR), but not limited to Polyisoprene Rubber, Polychloroprene Rubber, Polyvinyl Chloride, and the like.

In accordance with an embodiment of the present invention, a rubber article prepared by using nano size particles includes, latex from a latex source; and a compounded latex produced by mixing the latex with rubber chemicals. In use, the rubber article is prepared via an article dipping or a molding process by using the compounded latex. Essentially, the rubber article is a rubber glove. Those of ordinary skills in the art will appreciate that the present disclosure and various embodiments of the present invention are disclosed herein in a manner such that the novel and unique aspects may be implemented for rubber gloves, different types of one or more rubber articles, and the like. In accordance with an embodiment of the present invention, the latex source is natural rubber latex. In use, the latex source may be synthetic latex as well.

In accordance with an embodiment of the present invention, the rubber chemicals include a plurality of nano size particles of an active substance. Also, the rubber article is prepared via the step of post-processing the rubber article with or without surface retreatment to enhance surface morphologies for better handling of instruments. Furthermore, as disclosed hereinabove, the rubber article is prepared via the step of using the plurality of nano size particles with the an active substance onto the rubber article to yield a compact structure of a rubber matrix. Additionally, the rubber article is further prepared via the step of infusing nanoparticles active substances into a rubber matrix which contains one or at least one or combination of a plurality of ingredients, such as alkanolamines, amino acids, surfactants, plant-based derivatives in a base of colloids, micelles to give anti-microbial properties, and the like.

In accordance with an embodiment of the present invention, the rubber chemicals are selected from a group including at least one or combination of chemicals including Sulphur, Zinc Oxide, ZDEC, ZDBC, any other curative chemicals, crossliner, antioxidant, colorant, and the like. Also, the rubber article is produced in a manner such that the nanoparticles active substances provide effective electrostatic resistance.

In accordance with an embodiment of the present invention, the rubber article is produced in a manner such that the nanoparticles active substances provide effective heat protection.

In accordance with an embodiment of the present invention, the rubber article is produced in a manner such that the nanoparticles active substances provide effective chemo drug resistance.

In accordance with an embodiment of the present invention, the rubber article is produced with nanoparticles active substances by using polymer including Natural Rubber Latex and/or Synthetic Latex, such as Nitrile Butadiene Rubber (NBR), but not limited to Polyisoprene Rubber, Polychloroprene Rubber, Polyvinyl Chloride, and the like.

### DESCRIPTION OF BEST MODE

Those of ordinary skills in the art will appreciate that the embodiments of the instant invention may be embodied through various types of rubber articles, such as, for example, but not limited to, rubber gloves, and the like.

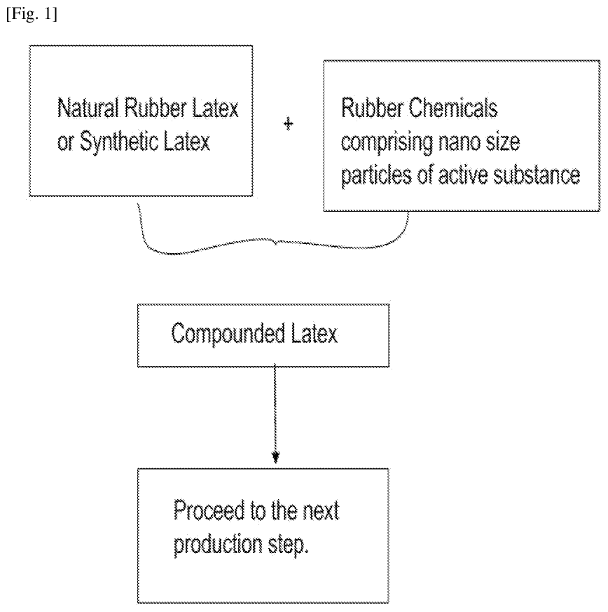

FIG. 1 illustrates a conceptual level flow diagram for preparing a rubber article by using nano size particles.

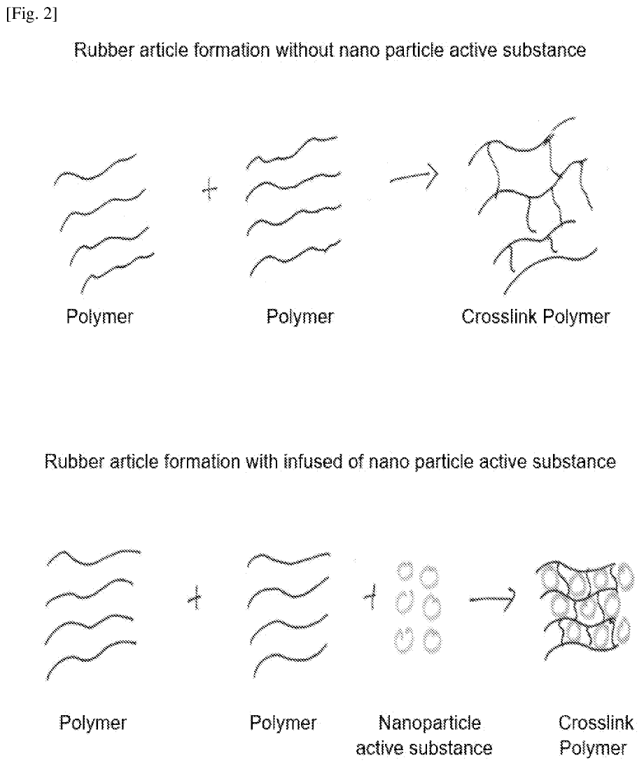

FIG. 2 illustrates a conceptual level illustration of the rubber articles or gloves infused with nanoparticle active substance.

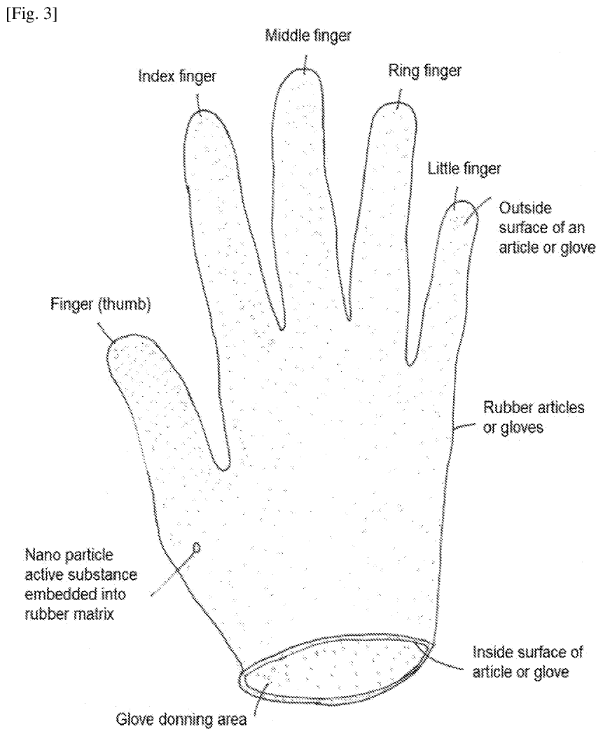

FIG. 3 illustrates rubber articles or gloves consist of nanoparticles' active substances, in accordance with an embodiment of the present invention.

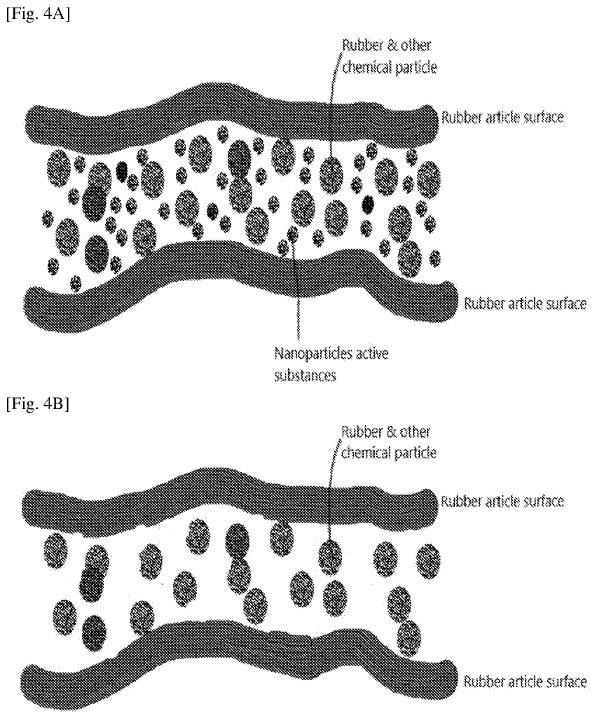

FIG. 4A and FIG. 4B illustrate a comparative illustration of rubber articles or gloves with and without nanoparticle active substance infused into the matrix. Specifically,

FIG. 4A rubber matrix shows that the rubber matrix formation is more compact compared to the FIG. 4B rubber matrix.

As disclosed herein, multiple embodiments of the instant invention of nanoparticle active substances into present articles or gloves improves the properties of the articles or gloves on film strength.

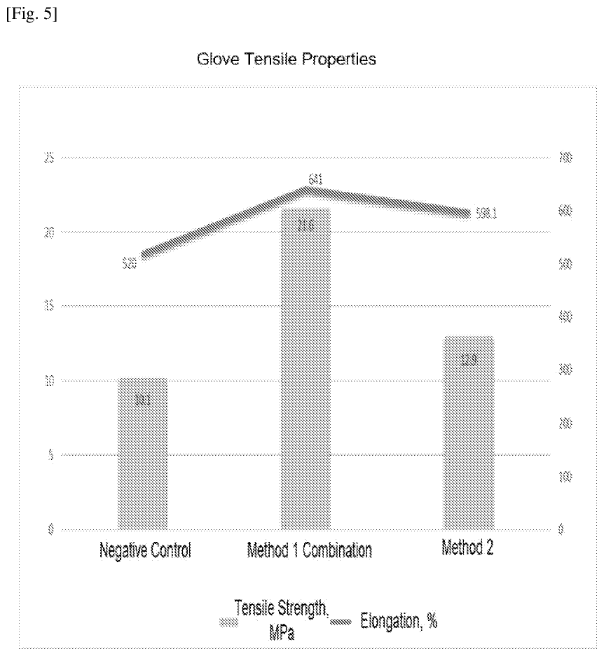

FIG. 5 illustrates a graphical illustration, wherein glove tensile properties comparison between article without nanoparticle active substance (Negative Control) against article with nanoparticle active substance (via Method 1 and Method 2 combination) is illustrated. As shown therein, the Graph 1 illustrates that rubber articles or gloves with nanoparticle active substances infused into the rubber matrix (via Method 1 and Method 2) yield better tensile strength of about 20% or above improvement compared to rubber articles or gloves without nanoparticle active substances infused into the rubber matrix (Negative Control). In use, both Method 1 and Method 2 articles or gloves yield Tensile Strength of 21.6 MPa and 12.9 MPa respectively against Negative Control of 10.1 MPa. The film has shown the improvement in elongation where both Method 1 and Method 2 articles or gloves yield 641% and 598.1% respectively compared to 520% elongation for Negative Control. Accordingly, it can be stated that the present articles or gloves with nanoparticles with active substances provide the compact molecular structure compared to articles or gloves without nanoparticles active substances.

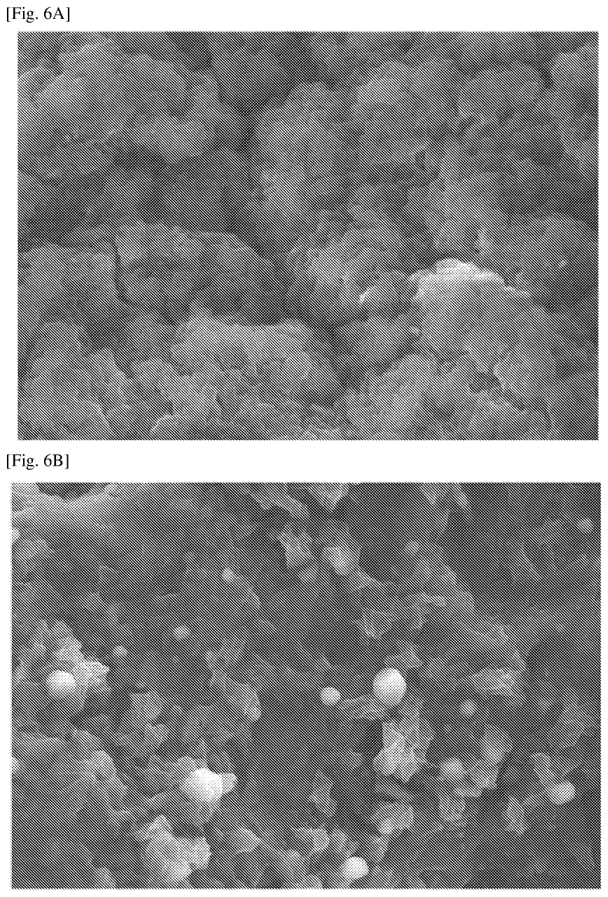

FIG. 6A illustrates an image of the rubber articles or gloves without the nanoparticle active substance, Negative Control; and FIG. 6B illustrates an image of rubber articles or gloves with nanoparticle active substance, via Method 1 or Method 2.

In use, FIG. 6A and FIG. 6B demonstrated the comparison of rubber articles or gloves morphologies without nanoparticle active substance (negative control) and with nanoparticles active substance (via method 1 and method 2). Also, the rubber article or glove surface in FIG. 6A shows uneven rubber surface morphology with a gap in between the bonding, whereas rubber articles or glove surface in FIG. 6B shows the nanoparticles active substance is embedded onto the rubber matrix to form compact or strong bonding.

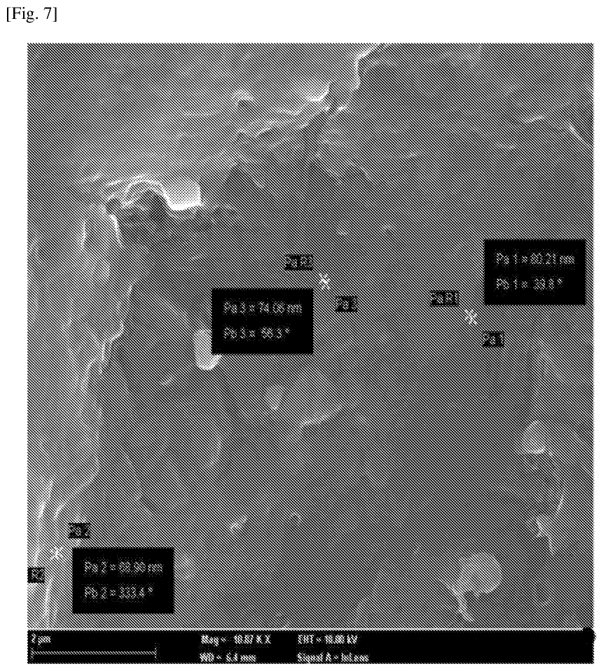

FIG. 7 illustrates the particle size of nanoparticles with active substances embedded into rubber matrix. As shown therein, FIG. 7 shows that rubber articles or gloves produced from Method 1 or Method 2 comprising nanoparticle with active substances where particles size are ranging between 60-80 nm.

In addition, the invention of rubber articles or gloves comprising nanoparticles active substances infused into rubber matrix which containing one or at least one or combination of the ingredients such as alkanolamines, amino acids, surfactants, plant-based derivatives in a base of colloids, micelles gave the anti-microbial properties. The products as disclosed herein have an effective kill rate on microorganisms such as Escherichia coli, Staphylococcus Aureus, Pseudomonas Aeruginosa, but not limited to Covid virus. The nanoparticles active substance has shown better durability in kill rate where the result of Inoculum count is maintained <10 cfu/mL after 0 min, 5 min, 15 min and 30 min contact with the tested organism. Accordingly, the test report as per tabulated below, wherein the rubber articles or gloves comprising nanoparticles that infused into the rubber matrix can retain the antimicrobial properties upon one to two times of wash under running water.

### Results of Analysis:

| | | Contact Time: 0 min | | Contact Time: 5 min | |
|---|---|---|---|---|---|
| | Innoculum | | Reduction | | Reduction |
| Test Organism | Count, CFU/mL | Result, CFU/mL | Percentage, % | Result, CFU/mL | Percentage, % |
| Escherichia coli | 4.3 × 10⁵ | <10 | >99.9 | <10 | >99.9 |
| ATCC 8739 | | | | | |
| Staphylococcus aureus | 4.0 × 10⁵ | <10 | >99.9 | <10 | >99.9 |
| ATCC 6538 | | | | | |
| Pseudomonas aeruginosa | 2.7 × 10⁵ | <10 | >99.9 | <10 | >99.9 |
| ATCC 9027 | | | | | |

| | |
|---|---|
| Uninocculated Sample | No Growth |

Method: In House USP 51

### Results of Analysis:

| | | Contact Time: 15 min | | Contact Time: 30 min | |
|---|---|---|---|---|---|
| | Innoculum | | Reduction | | Reduction |
| Test Organism | Count, CFU/mL | Result, CFU/mL | Percentage, % | Result, cfu/ml | Percentage, % |
| Escherichia coli | 4.3 × 10⁵ | <10 | >99.9 | <10 | >99.9 |
| ATCC 8739 | | | | | |
| Staphylococcus aureus | 4.0 × 10⁵ | <10 | >99.9 | <10 | >99.9 |
| ATCC 6538 | | | | | |
| Pseudomonas aeruginosa | 2.7 × 10⁵ | <10 | >99.9 | <10 | >99.9 |
| ATCC 9027 | | | | | |

| | |
|---|---|
| Uninocculated Sample | No Growth |

Method: In House USP 51

As illustrated hereinabove, the compact molecular structure yields better and compact pore size which gives a good electrostatic resistivity, thermal insulation properties where the heat from external is redundant as well as protects against chemo-drug permeation.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” ‘having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith. Therefore, the appended claims are to be construed to cover all equivalents falling within the true scope and spirit of the invention.

1. A method for preparing a rubber article by using nano size particles, said method comprising the steps of:
providing latex from a latex source;
mixing said latex with rubber chemicals to produce a compounded latex; and,
using said compounded latex to prepare said rubber article via an article dipping or a molding process.

2. The method as claimed in claim 1, wherein said rubber article is a rubber glove.

3. The method as claimed in claim 1, wherein said latex source is natural rubber latex.

4. The method as claimed in claim 1, wherein said latex source is synthetic latex.

5. The method as claimed in claim 1, wherein said rubber chemicals comprise a plurality of nano size particles of an active substance.

6. The method as claimed in claim 1, wherein said method further comprises the step of post-processing said rubber article with or without surface retreatment to enhance surface morphologies for better handling of instruments.

7. The method as claimed in claim 5, wherein said method further comprises the step of using said plurality of nano size particles with said active substance onto said rubber article to yield a compact structure of a rubber matrix.

8. The method as claimed in claim 7, wherein said method further comprises the step of infusing nanoparticles active substances into a rubber matrix which contains one or at least one or combination of a plurality of ingredients, such as alkanolamines, amino acids, surfactants, plant-based derivatives in a base of colloids, micelles to give anti-microbial properties, and the like.

9. The method as claimed in claim 1, wherein said rubber chemicals are selected from a group comprising at least one or combination of chemicals comprising Sulphur, Zinc Oxide, ZDEC, ZDBC, any other curative chemicals, crossliner, antioxidant, colorant, and the like.

10. The method as claimed in claim 1, wherein said method further comprises the step of producing said rubber article with nanoparticles active substances leading to effective electrostatic resistance.

11. The method as claimed in claim 1, wherein said method further comprises the step of producing said rubber article with nanoparticles active substances leading to effective heat protection.

12. The method as claimed in claim 1, wherein said method further comprises the step of producing said rubber article with nanoparticles active substances leading to effective chemo drug resistance.

13. The method as claimed in claim 1, wherein said method further comprises the step of producing said rubber article with nanoparticles active substances by using polymer including Natural Rubber Latex and/or Synthetic Latex, such as Nitrile Butadiene Rubber (NBR), but not limited to Polyisoprene Rubber, Polychloroprene Rubber, Polyvinyl Chloride, and the like.

14. A rubber article prepared by using nano size particles, said rubber article comprising:
latex from a latex source; and
a compounded latex produced by mixing said latex with rubber chemicals; characterized in that said rubber article is prepared via an article dipping or a molding process by using said compounded latex.

15. The rubber article as claimed in claim 14, wherein said rubber article is a rubber glove.

16. The rubber article as claimed in claim 14, wherein said latex source is natural rubber latex.

17. The rubber article as claimed in claim 14, wherein said latex source is synthetic latex.

18. The rubber article as claimed in claim 14, wherein said rubber chemicals comprise a plurality of nano size particles of an active substance.

19. The rubber article as claimed in claim 14, wherein said rubber article is prepared via the step of post-processing said rubber article with or without surface retreatment to enhance surface morphologies for better handling of instruments.

20. The rubber article as claimed in claim 18, wherein said rubber article is prepared via the step of using said plurality of nano size particles with said an active substance onto said rubber article to yield a compact structure of a rubber matrix.

21. The rubber article as claimed in claim 20, wherein said rubber article is further prepared via the step of infusing nanoparticles active substances into a rubber matrix which contains one or at least one or combination of a plurality of ingredients, such as alkanolamines, amino acids, surfactants, plant-based derivatives in a base of colloids, micelles to give anti-microbial properties, and the like.

22. The rubber article as claimed in claim 14, wherein said rubber chemicals are selected from a group comprising at least one or combination of chemicals comprising Sulphur, Zinc Oxide, ZDEC, ZDBC, any other curative chemicals, crossliner, antioxidant, colorant, and the like.

23. The rubber article as claimed in claim 14, wherein said rubber article is produced in a manner such that said nanoparticles active substances provide effective electrostatic resistance.

24. The rubber article as claimed in claim 14, wherein said rubber article is produced in a manner such that said nanoparticles active substances provide effective heat protection.

25. The rubber article as claimed in claim 14, wherein said rubber article is produced in a manner such that said nanoparticles active substances provide effective chemo drug resistance.

26. The rubber article as claimed in claim 14, wherein said rubber article is produced with nanoparticles active substances by using polymer including Natural Rubber Latex and/or Synthetic Latex, such as Nitrile Butadiene Rubber (NBR), but not limited to Polyisoprene Rubber, Polychloroprene Rubber, Polyvinyl Chloride, and the like.
